(19) United States
(12) Patent Application Publication
(10) Pub. No.: US 2024/0177814 A1
(43) Pub. Date: May 30, 2024

(54) TEST RESULT PROCESSING AND STANDARDIZATION ACROSS MEDICAL TESTING LABORATORIES

(71) Applicant: Flatiron Health, Inc., New York, NY (US)

(72) Inventors: Alexandre Costet, New York, NY (US); Elena Ruyter, New York, NY (US); Haley Spitz, Miami, FL (US); Nathaniel Wade, Rochester, NY (US); Beth Crane, New York, NY (US); Kim Peifer, Philadelphia, PA (US); Eric Stephens, New York, NY (US); Emma Vail Cangialosi, New York, NY (US); Murat Sincan, Sioux Falls, SD (US)

(21) Appl. No.: 18/522,949

(22) Filed: Nov. 29, 2023

Related U.S. Application Data
(60) Provisional application No. 63/429,011, filed on Nov. 30, 2022.

(51) Int. Cl. G16H 10/40; G16B 45/00; G16H 10/60; G16H 15/00; G16H 50/20

(52) U.S. Cl. CPC G16H 10/40; G16B 45/00; G16H 10/60; G16H 15/00; G16H 50/20

(57) ABSTRACT

A system for standardizing medical testing data may include a processor. The processor may be programmed to access a first medical testing record including a first data element represented in a first data format; access a second medical testing record including a second data element represented in a second data format, the second data format being different from the first data format; determine that the first data element and the second data element are associated with a common value classifier; and store the first data element and the second data element in a database in association with the common value classifier.

### CROSS REFERENCE TO RELATED APPLICATION

This application is based on and claims the benefit of priority of U.S. Provisional Application No. 63/429,011, filed on Nov. 30, 2022. The contents of the foregoing application are incorporated herein by reference in their entirety.

### BACKGROUND

### Technical Field

The present disclosure relates to the analysis of medical data and, more specifically, to parsing and mapping test data elements to a standardized value set.

### Background Information

In today's health care environment, genomic information plays an increasingly critical role in the diagnosis and treatment of cancers and various other patient conditions. In oncology, for example, genomic information may be used to provide more accurate diagnoses and treatment strategies that are tailored to particular aspects of a patient's tumor through a practice known as precision medicine. To provide this improved diagnosis and treatment, genomic data is often ordered through molecular profiling labs, and this genomic data may include profiles such as Whole Exome Sequencing (WES), Whole Genome Sequencing (WGS), Immunohisto-chemistry (IHC), Whole Transcriptome Sequencing (WTS).

Results from these profiles are generally returned from molecular profiling labs in the form of a portable document format (PDF), or other various other document formats. Some molecular profiling labs have also begun working with electronic health records (EHRs) to make genomic result data available in a standardized or structured format. However, unlike traditional laboratory data, there is not currently an industry-accepted standard for the structure of computable genomic results reporting. Rather, data received from different molecular profiling labs is structured differently with varying levels of terminology-coded data, creating a challenge in presenting data from different labs as discrete data in an EHR or various other applications.

As one example, researchers who wish to perform a statistical analysis on patient genomic data often use relatively large data sets (e.g., thousands, tens of thousands, hundreds of thousands, or millions of patients, or more) in order to draw meaningful insights from the data. The sheer volume of data that a researcher would have to review makes manual extraction of dates or other information infeasible. Computer-based extraction and data processing methods are often ineffective given the inconsistent and unstandardized formatting used among different molecular profiling labs.

Accordingly, in view of these and other deficiencies in current techniques, technical solutions are needed to standardize and harmonize genomic data from a variety of molecular profiling labs so that it can be surfaced in a clinically consistent and scalable manner.

### SUMMARY

Embodiments consistent with the present disclosure include systems and methods for standardizing medical testing data. In an embodiment, a system may comprise a least one processor. The processor may be programmed to access a first medical testing record including a first data element represented in a first data format; access a second medical testing record including a second data element represented in a second data format, the second data format being different from the first data format; determine that the first data element and the second data element are associated with a common value classifier; and store the first data element and the second data element in a database in association with the common value classifier.

In an embodiment, a method for standardizing medical testing data is disclosed. The method may include accessing a first medical testing record including a first data element represented in a first data format; accessing a second medical testing record including a second data element represented in a second data format, the second data format being different from the first data format; determining that the first data element and the second data element are associated with a common value classifier; and storing the first data element and the second data element in a database in association with the common value classifier.

In an embodiment, a system for standardizing molecular profiling data may comprise a least one processor. The processor may be programmed to access a molecular profiling record associated with a patient, the molecular profiling record including at least one genomic data element; access a data structure including a plurality of predefined genomic data classifiers; analyze the molecular profiling record to determine a correlation between the at least one genomic data element and a particular genomic data classifier of the predefined genomic data classifiers; convert the at least one genomic data element to a format associated with the particular genomic data classifier; cause display of a graphical user interface in association with the patient. The graphical user interface may include a representation of the at least one genomic data element in the format associated with the particular genomic data classifier and the graphical user interface may include an interactive element displayed in association with the representation of the at least one genomic data element for causing display of the molecular profiling record.

In an embodiment, a method for standardizing medical testing data is disclosed. The method may include accessing a molecular profiling record associated with a patient, the molecular profiling record including at least one genomic data element; accessing a data structure including a plurality of predefined genomic data classifiers; analyzing the molecular profiling record to determine a correlation between the at least one genomic data element and a particular genomic data classifier of the predefined genomic data classifiers; converting the at least one genomic data element to a format associated with the particular genomic data classifier; and causing display of a graphical user interface in association with the patient. The graphical user interface may include a representation of the at least one genomic data element in the format associated with the particular genomic data classifier and the graphical user interface may include an interactive element displayed in association with the representation of the at least one genomic data element for causing display of the molecular profiling record.

Consistent with other disclosed embodiments, non-transitory computer readable storage media may store program instructions, which are executed by at least one processor and perform any of the methods described herein.

### BRIEF DESCRIPTION OF THE DRAWINGS

The accompanying drawings, which are incorporated in and constitute part of this specification, and together with the description, illustrate and serve to explain the principles of various exemplary embodiments. In the drawings:

FIG. 1 is a block diagram illustrating an exemplary system environment for implementing embodiments consistent with the present disclosure.

FIG. 2A is a block diagram showing an example server, consistent with the disclosed embodiments.

FIG. 2B is a block diagram showing an example client device, consistent with the disclosed embodiments.

FIG. 3 is a block diagram illustrating an example process for mapping data elements to predefined classifiers, consistent with the disclosed embodiments.

FIG. 4 illustrates an example process for training and implementing a model for identifying data elements and mapping them to data classifiers, consistent with the disclosed embodiments.

FIG. 5 illustrates an example graphical user interface that may be generated, consistent with the disclosed embodiments.

FIG. 6 illustrates an example of an additional graphical user interface that may be generated, consistent with the disclosed embodiments.

FIG. 7 is a flowchart showing an example process for standardizing medical testing data, consistent with the disclosed embodiments.

FIG. 8 is a flowchart showing an example process 800 for standardizing molecular profiling data, consistent with the disclosed embodiments.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Embodiments disclosed herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, nonvolatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

The disclosed systems and methods may provide a vendor-agnostic, centralized database that harmonizes and standardizes molecular test results (or other forms of reports) from various vendors or sources (e.g., molecular profiling labs, etc.). As a result, critical data elements from across labs may be harmonized so that the information can be presented efficiently and impactfully at the point of care. For example, a first molecular profiling report received from a first vendor and a second molecular profiling report received from a second vendor may both include data elements associated with the same biomarker. However, the data from each of the first and second vendors may be presented in different formats, such that is not readily apparent which elements in the reports correspond to the same type of data element. However, as a result of the mapping process described herein, the corresponding data elements from each of the first and second molecular profiling reports may be coded consistently such that the data elements are coded with the same value. This coding and mapping process may be performed across each data element within a molecular profiling report, resulting in a database of consistently-structured molecular profiling data.

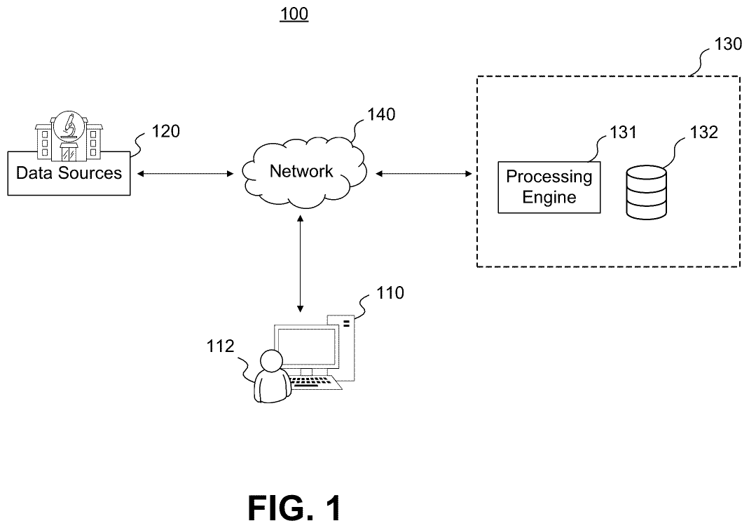

FIG. 1 illustrates an example system environment 100 for implementing embodiments consistent with the present disclosure, described in detail below. As shown in FIG. 1, system environment 100 may include several components, including client devices 110, data sources 120, system 130, and network 140. It will be appreciated from this disclosure that the number and arrangement of these components is exemplary and provided for purposes of illustration. Other arrangements and numbers of components may be used without departing from the teachings and embodiments of the present disclosure.

As shown in FIG. 1, exemplary system environment 100 may include a system 130. System 130 may include one or more server systems, databases, and/or computing systems configured to receive information from entities over a network, process the information, store the information, and display/transmit the information to other entities over the network. Thus, in some embodiments, the network may facilitate cloud sharing, storage, and/or computing. In one embodiment, system 130 may include a processing engine 131 and one or more databases 132, which are illustrated in a region bounded by a dashed line representing system 130. Processing engine 131 may comprise at least one processing device, such as one or more generic processors, e.g., a central processing unit (CPU), a graphics processing unit (GPU), or the like and/or one or more specialized processors, e.g., an application-specific integrated circuit (ASIC), a field-programmable gate array (FPGA), or the like, as described in further detail below.

Data transmitted and/or exchanged within system environment 100 may occur over a data interface. As used herein, a data interface may include any boundary across which two or more components of system environment 100 exchange data. For example, environment 100 may exchange data between software, hardware, databases, devices, humans, or any combination of the foregoing. Furthermore, it will be appreciated that any suitable configuration of software, processors, data storage devices, and networks may be selected to implement the components of system environment 100 and features of related embodiments.

The components of environment 100 (including system 130, client devices 110, and data sources 120) may communicate with each other or with other components through network 140. Network 140 may comprise various types of networks, such as the Internet, a wired Wide Area Network (WAN), a wired Local Area Network (LAN), a wireless WAN (e.g., WiMAX), a wireless LAN (e.g., IEEE 802.11, etc.), a mesh network, a mobile/cellular network, an enterprise or private data network, a storage area network, a virtual private network using a public network, a nearfield communications technique (e.g., Bluetooth, infrared, etc.), or various other types of network communications. In some embodiments, the communications may take place across two or more of these forms of networks and protocols.

System 130 may be configured to receive and store the data transmitted over network 140 from various data sources, including data sources 120, process the received data, and transmit data and results based on the processing to client device 110 (or otherwise make the data and results available to client device 110). For example, system 130 may be configured to receive patient data from data sources 120 or other sources in network 140. In some embodiments, the patient data may include medical information stored in the form of one or more medical testing records. Each medical testing record may be associated with a particular patient. In some embodiments, a medical testing record may include results or data associated with multiple patients. Data sources 120 may be associated with a variety of sources of medical information for a patient. For example, data sources 120 may include laboratories such as radiology or other imaging labs, hematology labs, pathology labs, etc. Data sources 120 may also be associated medical care providers of the patient, such as physicians, nurses, specialists, consultants, hospitals, clinics, and the like. In some embodiments, data sources 120 may also be associated with insurance companies or any other sources of patient data.

System 130 may further communicate with one or more client devices 110 over network 140. For example, system 130 may provide results based on analysis of information from data sources 120 to client device 110. Client device 110 may include any entity or device capable of receiving or transmitting data over network 140. For example, client device 110 may include a computing device, such as a server or a desktop or laptop computer. Client device 110 may also include other devices, such as a mobile device, a tablet, a wearable device (i.e., smart watches, implantable devices, fitness trackers, etc.), a virtual machine, an IoT device, or other various technologies. In some embodiments, client device 110 may access information about one or more patients over network 140 from system 130, such as medical test data associated with a particular patient. User endpoint device 110 may be configured such that a user 112 may access this medical test data through a browser or other software executing on user endpoint device 110. A user of system environment 100 may encompass any individual who may wish to access and/or analyze patient data. Thus, throughout this disclosure, references to a “user” of the disclosed embodiments may encompass any individual, such as a physician, a researcher, a quality assurance department at a health care institution, and/or any other individual.

The various components of system environment 100 may include an assembly of hardware, software, and/or firmware, including a memory, a central processing unit (CPU), and/or a user interface. Memory may include any type of RAM or ROM embodied in a physical storage medium, such as magnetic storage including floppy disk, hard disk, or magnetic tape; semiconductor storage such as solid-state disk (SSD) or flash memory; optical disc storage; or magneto-optical disc storage. A CPU may include one or more processors for processing data according to a set of programmable instructions or software stored in the memory. The functions of each processor may be provided by a single dedicated processor or by a plurality of processors. Moreover, processors may include, without limitation, digital signal processor (DSP) hardware, or any other hardware capable of executing software. An optional user interface may include any type or combination of input/output devices, such as a display monitor, keyboard, and/or mouse.

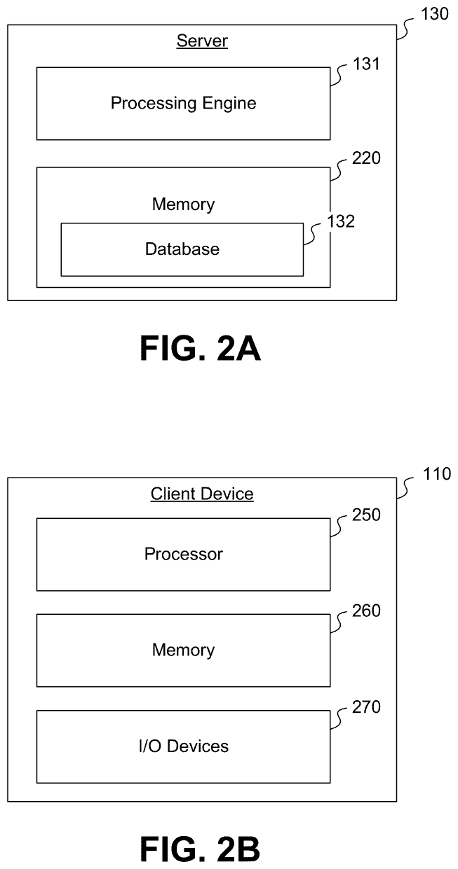

FIG. 2A is a block diagram showing an example system 130, consistent with the disclosed embodiments. As described above, system 130 may be a computing device (e.g., a server, etc.) and may include one or more dedicated processors and/or memories. For example, system 130 may include a processing engine (which may include a processor or multiple processors) 131, and a memory (or multiple memories) 220, as shown in FIG. 2A.

Processing engine 131 may take the form of, but is not limited to, a microprocessor, embedded processor, or the like, or may be integrated in a system on a chip (SoC). Furthermore, according to some embodiments, processing engine 131 may be from the family of processors manufactured by Intel®, AMD®, Qualcomm®, Apple®, NVIDIA®, or the like. The processing engine 131 may also be based on the ARM architecture, a mobile processor, or a graphics processing unit, etc. The disclosed embodiments are not limited to any type of processor configured in system 130.

Memory 220 may include one or more storage devices configured to store instructions used by processing engine 131 to perform functions related to system 130. The disclosed embodiments are not limited to particular software programs or devices configured to perform dedicated tasks. For example, memory 220 may store a single program, such as a user-level application, that performs the functions associated with the disclosed embodiments, or may comprise multiple software programs. Additionally, processing engine 131 may, in some embodiments, execute one or more programs (or portions thereof) remotely located from system 130. Furthermore, memory 220 may include one or more storage devices configured to store data for use by the programs. Memory 220 may include, but is not limited to a hard drive, a solid state drive, a CD-ROM drive, a peripheral storage device (e.g., an external hard drive, a USB drive, etc.), a network drive, a cloud storage device, or any other storage device.

In some embodiments, memory 220 may include a database 132 as described above. Database 132 may be included on a volatile or non-volatile, magnetic, semiconductor, tape, optical, removable, non-removable, or other type of storage device or tangible or non-transitory computer-readable medium. Database 132 may also be part of system 130 or separate from system 130. When database 132 is not part of system 130, system 130 may exchange data with database 132 via a communication link. Database 132 may include one or more memory devices that store data and instructions used to perform one or more features of the disclosed embodiments. Database 132 may include any suitable databases, ranging from small databases hosted on a work station to large databases distributed among data centers. Database 132 may also include any combination of one or more databases controlled by memory controller devices (e.g., server(s), etc.) or software. For example, database 132 may include document management systems, Microsoft SQL™ databases, SharePoint™ databases, Oracle™ databases, Sybase™ databases, other relational databases, or non-relational databases, such as mongo and others.

FIG. 2B is a block diagram showing an example client device 131, consistent with the disclosed embodiments. Client device 131 may correspond to one or both of user endpoint device 110 and auditor endpoint device 120. As shown in FIG. 2B, client device 131 may include a processor (or multiple processors) 250, a memory (or multiple memories) 260, and/or one or more input/output (I/O) devices 270, as shown in FIG. 2B.

As with processing engine 131, processor 250 may take the form of, but is not limited to, a microprocessor, embedded processor, or the like, or may be integrated in a system on a chip (SoC). Furthermore, according to some embodiments, processor 250 may be from the family of processors manufactured by Intel®, AMD®, Qualcomm®, Apple®, NVIDIA®, or the like. The processor 250 may also be based on the ARM architecture, a mobile processor, or a graphics processing unit, etc. The disclosed embodiments are not limited to any type of processor configured in client device 131.

Further, similar to memory 220, memory 260 may include one or more storage devices configured to store instructions used by the processor 250 to perform functions related to client device 131. The disclosed embodiments are not limited to particular software programs or devices configured to perform dedicated tasks. For example, memory 260 may store a single program, such as a user-level application (e.g., a browser), that performs the functions associated with the disclosed embodiments, or may comprise multiple software programs. Additionally, processor 250 may, in some embodiments, execute one or more programs (or portions thereof) remotely located from client device 131 (e.g., located on system 130). Furthermore, memory 260 may include one or more storage devices configured to store data for use by the programs. Memory 260 may include, but is not limited to a hard drive, a solid state drive, a CD-ROM drive, a peripheral storage device (e.g., an external hard drive, a USB drive, etc.), a network drive, a cloud storage device, or any other storage device.

I/O devices 270 may include one or more network adaptors or communication devices and/or interfaces (e.g., WIFI, BLUETOOTH, RFID, NFC, RF, infrared, Ethernet, etc.) to communicate with other machines and devices, such as with other components of system environment 100 through network 140. For example, client device 131 may use a network adaptor to receive and transmit communications pertaining to medical testing records within system environment 100. In some embodiments, I/O devices 270 may also include interface devices for interfacing with a user of client device 131, such as user 112 or 122. For example, I/O devices 270 may comprise a display, touchscreen, keyboard, mouse, trackball, touch pad, stylus, printer, or the like, configured to allow a user to interact with client device 131.

In some embodiments, system 130 may be configured to analyze medical testing record (or other forms of medical data) to identify data elements within the medical testing record. For example, system 130 may receive a molecular profiling record associated with a patient from a medical testing lab and may analyze the molecular profiling record to identify at least one genomic data element represented in the molecular profiling record. System 130 may similarly be configured to identify various other genomic data elements represented in molecular profiling records from other medical testing labs. In many cases, however, data received from different labs is structured differently with varying levels of terminology-coded data, creating a challenge in presenting data as discrete data in an EHR or various other applications. Accordingly, system 130 may be configured to standardize and harmonize genomic data from a variety of molecular profiling labs so that it can be surfaced in a clinically consistent and scalable manner.

In order to efficiently extract and harmonize this genomic data, a mapping process may be performed. This may include receiving genomic data from a plurality of vendors or a plurality of entities. In some embodiments, the genomic data may be represented in a genomic data report, which may include either or both structured and unstructured genomic data associated with a patient. For example, the genomic data report may be presented in a pdf document including structured data and/or unstructured data. In some embodiments, the genomic data report may be presented in the form of multiple documents. For example, a pdf may include unstructured data associated with the patient and may be accompanied with additional structured data in a JavaScript Object Notation (JSON) or similar format. As used herein, structured data may include quantifiable or classifiable data about the patient. In terms of molecular profiling for oncology patients, this may include results of various biomarker testing associated with DNA, RNA, or proteins of a patient. In some embodiments, this may include various other patient data, such as gender, age, race, weight, vital signs, lab results, date of diagnosis, diagnosis type, disease staging (e.g., billing codes), therapy timing, procedures performed, visit date, practice type, insurance carrier, medication orders, medication administrations, or any other measurable data about the patient. Unstructured data may include information about the patient that is not quantifiable or easily classified, such as descriptions or characterizations of a patient's molecular profiling.

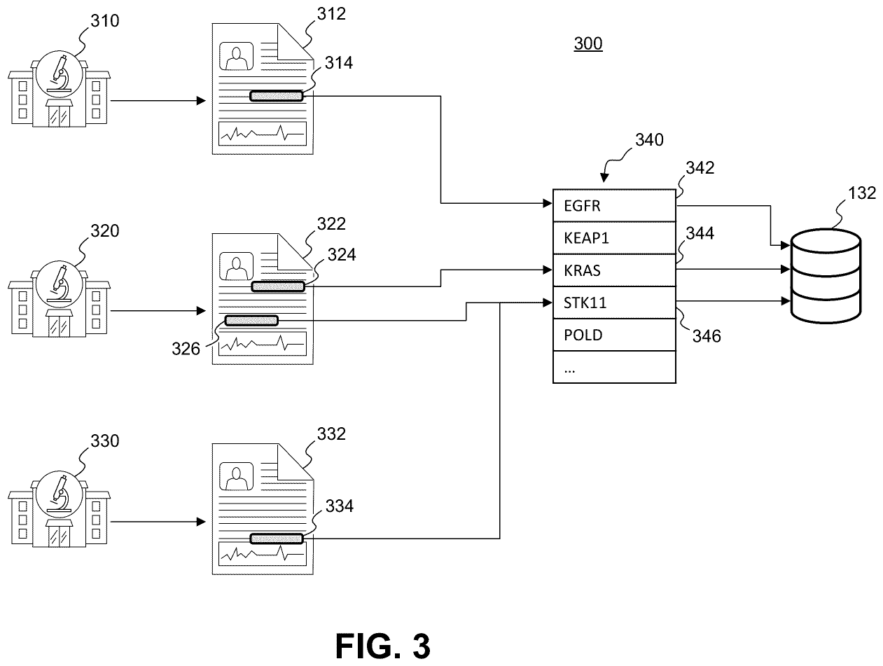

FIG. 3 is a block diagram illustrating an example process 300 for mapping data elements to predefined classifiers, consistent with the disclosed embodiments. The structured and/or unstructured information received from various medical testing labs (e.g., molecular profiling labs or various other tabs) may be mapped to a common set of values. For example as shown in FIG. 3, system 130 may receive medical testing record 312 from medical testing lab 310. Similarly, system 130 may receive medical testing record 322 from medical testing lab 320 and medical testing record 332 from medical testing lab 330. Medical testing labs 310, 320, and 330 may correspond to data sources 120 described above.

In some embodiments, data may be received from medical testing labs 310, 320, and or 330 in a JSON format or another at least partially standardized format. For example, medical testing records 312, 322, and 332 may be presented as structured data in a JSON format. Despite being presented as structured data, the information may nonetheless be presented in a nonuniform manner, thereby presenting the difficulties in ingesting and interpreting this data described above. For example, medical testing labs 310, 320, and 330 may each present information in a JSON or other format, but each molecular profiling lab may represent the data in different ways. As an illustrative example, when reporting the detection of a particular biomarker, a first molecular profiling lab may include a field within a JSON format associating the biomarker with a “detected” attribute. Conversely, a second molecular profiling lab may represent the same result as a binary value within a particular field, such as with either a “1” or “0” indicating whether the biomarker was detected. Further, the data from different molecular profiling labs may be arranged differently such that the same or similar values are found in different locations within the genomic data report.

Accordingly, this received data may be mapped to a common set of values, as described above. For example, each data element within the genomic data report may refer to a different biomarker or other test results performed as part of a molecular profile. Each data element may be mapped to a corresponding value in a set of predefined values. System 130 be configured such that similar data elements represented in various molecular profiling reports are mapped similarly.

As shown in FIG. 3, medical testing records 312, 322, and 332 may include various data elements. For example, medical testing record 312 may include data element 314, medical testing record 322 may include data elements 324 and 326, and medical testing record 332 may include data element 334. As used herein, a data element may refer to any piece of information within a medical testing record. For example, a data element may include and indication of whether a patient tested positive or negative for a particular patient characteristic. In the example of molecular profiling reports used throughout the present disclosure, a data element may be a genomic data element and may represent, for example, whether a patient tested positive for a particular genomic biomarker. In other examples, a data element may be a particular laboratory test result value. For example, if medical testing record 312 is a blood test result, data element 314 may be a value indicating a patient has a creatine level of 0.9 mg/dL. Alternatively or additionally, data element 314 may be a value indicating whether the patient's creatine level is low, within a predefined reference range, or high. As another example, data element 314 may represented in a semantic or unstructured format, such as an explanation that a “the patient's creatine levels were normal” or “the patient's screening panel showed a creatine level of 0.9 mg/dL.”

System 130 may be configured to parse medical testing records 312, 322, and 332 to identify data elements 314, 324, 326, and 334. Process 300 may include mapping data elements 314, 324, 326 and 334 to one or more predefined values within a database. For example, system 130 may access a data structure 340 including a plurality of predefined data classifiers, as shown in FIG. 3. In this example, data structure 340 may include a plurality of predefined genomic data classifiers, such as specific biomarkers a patient may have been tested for. System 130 may be configured to identify data elements 314, 324, 326, and 334 within medical testing records 312, 322, and 332 and map data elements 314, 324, 326, and 334 to particular data classifiers within data structure 340. For example, system 130 may map data element 314 to data classifier 342, which may indicate whether a patient exhibits an epidermal growth factor receptor (EGFR) gene mutation, as shown in FIG. 3. In some embodiments, system 130 may be configured to extract multiple data elements from the same medical testing record. For example, as shown in FIG. 3, system 130 may map data element 324 in medical testing record 322 to data classifier 344, and may map data element 326 in medical testing record 322 to data classifier 346 which may indicate whether a patient exhibits KRAS and STK11 gene mutations, respectively. Accordingly, process 300 may include parsing a medical testing record for a plurality of different data elements and mapping them to classifiers within data structure 340.

Further, in some embodiments, system 130 may be configured to standardize data across multiple medical testing records. For example, like medical testing record 322, medical testing record 332 may include a data element 334 indicating whether a patient exhibits a STK11 gene mutation, and thus system 130 may map both data element 326 in medical testing record 322 and data element 334 in medical testing record 342 to data classifier 346. In some embodiments, medical testing record 322 and medical testing record 342 may be presented in different formats. For example, data element 326 may be presented in a format of either “1” or “0” in a particular field indicating whether the patient presents with a STK11 gene mutation, whereas data element 334 may be presented in a format of “STK11:neg” indicating the patient has tested negative. Accordingly, system 130 may be configured to recognize a wide variety of data formats and may identify and map potential data elements regardless of the format.

Process 300 may further include storing the mapped data elements in a database or other data structure. For example, data elements 314, 324, 326, and 334, once mapped to particular classifiers in data structure 340, may be stored in database 132, as shown. In some embodiments, data elements 314, 324, 326, and 334 may be stored in association with a particular patient. Accordingly, using process 300, a comprehensive catalog of data elements (e.g., test result values) for a particular patient may be compiled. In some embodiments, process 300 may further include determining an identity of a patient based on a medical testing report. For example, this may include extracting a patient name, a patient medical ID number, a social security number, a phone number, or various other forms of identifiers from medical testing records 312, 322, and 332. These patient identifiers may be extracted similar to other data elements, as described herein.

In some embodiments, process 300 may further include converting data elements 314, 324, 326, and 334 to a standardized format. Accordingly, all data elements mapped to data classifier 342 may be stored in database 132 in a common format, regardless of the format in which they appear in a medical testing record. Accordingly, some or all of the classifiers within data structure 340 may be associated with a predefined data format and any data elements mapped to a particular classifier may be converted to the predefined data format.

According to some embodiments, system 130 may be configured to store other information associated with a patient, medical testing labs 310, 320, and 330, and/or medical testing records 312, 322, and 332 in database 132. In some embodiments, data indicating how the mappings were generated and/or applied may be stored to allow the extraction of one or more parameters to be traced. For example, system 130 may be configured to store an indication of a particular medical testing record from which a data element was extracted. For example, data element 314 may be stored in database 130 in a manner such that it is associated with medical testing report 312 and/or medical testing lab 310. In some embodiments, medical testing report 312 may also be stored in database 132 or another storage location, such as memory 220. Accordingly, when viewing data associated with a patient, a medical testing report associated with a particular data element may be retrieved and presented to a user based on database 132. Consistent with some embodiments, system 130 may be further configured to store an indication of a location within a medical testing record that a data element appears. For example, this may include a particular page in which a data element appears, a location within a page the data element was found (e.g., represented in page coordinates, line numbers or ranges, etc.), or the like. Accordingly, when a medical testing report associated with a particular data element is retrieved and presented to a user, only a relevant portion of the medical testing report may be presented (e.g., a particular page or range of pages, a particular portion of a page, etc.), the data element may be highlighted within the medical testing report (e.g., by adding a bounding box, a highlighting color, etc.), or the like.

As another example, this may include generating and storing a data structure associated with a particular medical testing record and its mappings. In some embodiments, the data structure may include multiple columns indicating the mapping progression. For example, the data structure may include an ingestion version column showing the as-reported data, along with an additional column showing the predefined values the as-reported data is mapped to. As mappings are modified or changed, additional columns may be added to the data structure to reflect the updated mappings of values. Accordingly, any extracted data value may be traced back to the original source within a genomic data report, as well as to any previous versions of the mapping that are generated.

Data elements 314, 324, 326, and 334 may be identified within medical testing records 312, 322, and 332 in various ways. In some embodiments, parsing medical testing records 312, 322, and 332 may include performing an optical character recognition (OCR) or similar processing techniques to identify alphanumerical characters within the medical testing records. In some embodiments, data elements may be identified by locating words, phrases, symbols, abbreviations, or other information within a medical testing record that may indicate an association with a particular classifier in data structure 340. For example, data classifier 342 may be associated with a list of predefined terms associated with data classifier 342, and data element 314 may be identified by searching medical testing record 312 for one or more of these terms.

In some embodiments, once a particular format has been identified for representing data elements in a medical testing report, system 130 may store an indication of the format in association with the medical testing lab. For example, if system 130 identifies data element 314 as being represented in medical testing record 312 in the form “EGFR(+),” where the “+” indicates a positive test result, system 130 may store this format in association with medical testing lab 310. Accordingly, when parsing another medical testing record from medical testing lab 310, system 130 may at least initially search for data in the form “EGFR([ ])” (where “[ ]” represents a value for data element 314). As another example, a particular field or position within a medical testing report may be associated with a particular classifier. Accordingly, system 130 may automatically and continuously build a “template” for extracting values from medical testing reports from a particular medical testing lab. Once a mapping has been established for a particular lab, data from within a JSON data format (or other format) may be extracted and analyzed automatically based on the mapping.

Consistent with the disclosed embodiments, process 300 may further include determining a degree of confidence associated with a data element mapping. For example, system 130 may generate a score indicating a confidence that a particular data element corresponds to a value classifier. In some embodiments, the score may be based on how closely a format for a data element found in a medical testing record matches a known format. For example, if a format of “EGFR—positive” is associated with classifier 346, data element 326 represented as “EGFR-positive” may receive a higher confidence score than data element 334 represented as “EGFR_+,” as the format for data element 326 may be considered closer to the known format. In some embodiments, a confidence score may be based on a previous format associated with a particular data source. For example, as described above, medical testing lab 310 may be associated with a format of “EGFR([ ]).” A data element identified in the same format in a subsequent medical testing record received from medical testing lab 310 may be associated with a degree of confidence based on a number of previous data elements associated with the classifier represented in this same format. The confidence scores may be used by system 130 in various ways. In some embodiments, confidence scores may be assigned to a particular data element in association with multiple value classifiers. For example, a confidence score for data element 314 may be determined for each of classifiers 342, 344, and 346, and data element 314 may be assigned a value classifier based on which of the confidence scores is highest. As another example, a data element within a medical testing report may be associated with a classifier only if a confidence score exceeds a predefined threshold confidence value.

As a result of this coding and mapping process, data from various reports (including reports in various formats from different vendors) may be mapped to corresponding values within database 130. This data may be stored such that it is easily indexed, searched, or otherwise accessed from various entities. In some embodiments, an application programming interface (API) may be provided for accessing the data. This API may be used by various applications to access data stored in database 130. For example, these applications may include electronic health record applications, trial matching tool applications, and clinical decision support applications, or the like, which may be executed on client device 110.

The embodiments disclosed herein may include various other aspects to improve the standardization and harmonization of medical testing data. In some embodiments, this may include generating various alerts association with mapping the received data to a common set of values. In some embodiments, not all elements within a report may be mapped to the common set of values. For example, the system may identify one or more terms or values within a JSON file that are unrecognized, contain an error, or otherwise cannot be correlated to a predetermined value. Accordingly, a report or other notification may be generated identifying the unharmonized data. As another example, a JSON file may be missing an expected field and the missing field may be flagged or otherwise reported to indicate the inconsistency. In some embodiments, the genomic data report may be processed in addition to generating the report. For example, any terms within the JSON file that are mapped and/or harmonized may be processed despite the inclusion of unharmonized or missing terms to ensure the data is available for processing as soon as possible.

According to some embodiments, a trained machine learning model may be developed to parse and categorize data within a report. For example, a training set of raw molecular profiling data (which may include structured data, unstructured data, or both) may be input into a machine learning model. This training data may be labeled such that elements within the training data are mapped to various values within a predefined dataset. For example, mappings may be developed based on a training set of genomic data reports, and these mappings along with the raw genomic data reports may be input into a machine learning algorithm. Accordingly, a model may be trained to automatically generate similar mappings from various molecular profiling data reports. In some embodiments, the model may further be trained to automatically extract values in a consistent manner (with or without performing the intermediate step of developing a mapping), regardless of format or vendor.

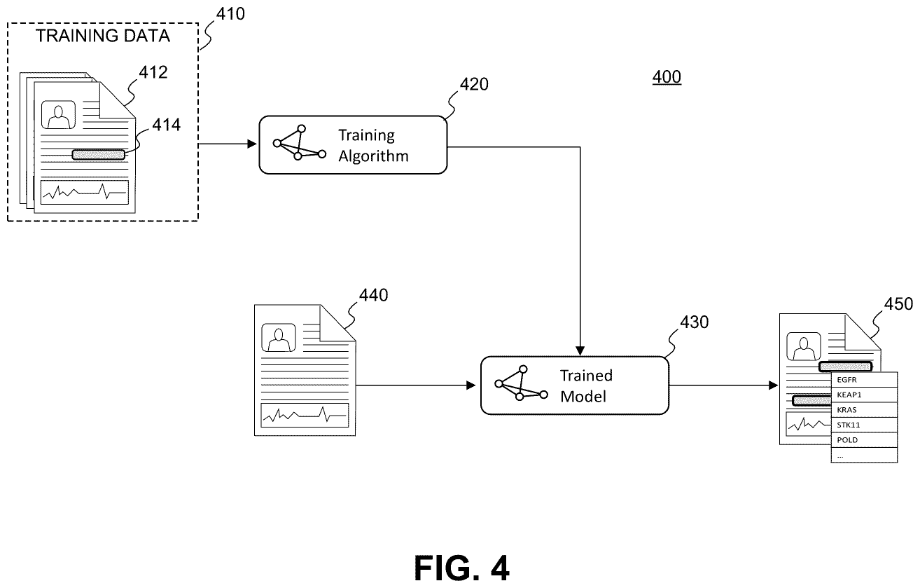

FIG. 4 illustrates an example process 400 for training and implementing a trained model 430 for identifying data elements and mapping them to data classifiers, consistent with the disclosed embodiments. Trained model 430 may be trained using a set of training data 410, which may be input into a training algorithm 410, as indicated in FIG. 4. Training data 410 may include at least one training medical test record, such as training medical test records 412, which may be used for training trained model 430. Training medical test records 412 may include any form of medical records that may include data elements extractable by system 130. For example, training medical test record 412 may be similar to medical testing records 312, 322, and 332 described above. Accordingly, training medical test records 412 may include various data elements and may be presented in various formats, as described herein.

In some embodiments, the training medical test records 412 may be labeled to indicate data elements within medical test records 412. For example, training medical test records 412 may be labeled to indicate data element 414 and various other data elements and classifiers for these data elements. Accordingly, through inputting the labeled records into training algorithm 412, trained model 430 may identify and classify data elements within other medical records.

Accordingly, as shown in FIG. 4, training data 410 may be input into training algorithm 420 to generate trained model 430. Training algorithm 410 may be any form of machine learning algorithm for generating trained model 430. In some embodiments, trained model 430 may include an artificial neural network, such as convolutional neural network (CNN). Various other machine learning algorithms may be used, including a logistic regression, a linear regression, a regression, a random forest, a K-Nearest Neighbor (KNN) model, a K-Means model, a decision tree, a cox proportional hazards regression model, a Naïve Bayes model, a Support Vector Machines (SVM) model, a gradient boosting algorithm, or any other form of machine learning model or algorithm. Through the training process, training algorithm 410 may recognize various data elements within training medical test records 412 and may correlate these various data elements with the labeled classifiers.

As a result of the training process, trained model 430 may be configured to receive one or more medical testing records as an input and generate as an output an indication of a mapping between data elements represented in the one or more medical testing records and one or more predefined value classifiers. For example, process 400 may include inputting medical test record 440 into trained model 430, as shown in FIG. 4 to generate an output 450. Output 450 may include information identifying data elements in medical test record 440 and their classifications, similar to the process described with respect to FIG. 3. Accordingly, the disclosed embodiments may be scalable such that new data sources may be integrated without remapping or redesigning the existing system. For example, if a new format of medical test record 440 is provided, trained model 430 may nonetheless be able to extract data elements and map them to predefined data classifiers without having to be reconfigured for the new data format.

In some embodiments, one or more graphical user interfaces may be generated to allow a user to view data extracted from one or more genomic data reports using the various processing and standardization techniques described herein. Accordingly, the disclosed embodiments may automatically extract data and surface the extracted data to a user. In some embodiments, the graphical user interface may allow a user to navigate data extracted from multiple genomic data reports from multiple molecular profiling labs. As described above, this data may not be readily accessible for processing using conventional techniques as it is not presented in any standardized format. Accordingly, users reviewing these reports would be limited to viewing individual reports (e.g., pdf files, etc.). However, the techniques for automatically extracting data from unstandardized documents described herein allow data from multiple molecular profiling labs to be presented in a combined manner, thus providing an improvement over existing techniques.

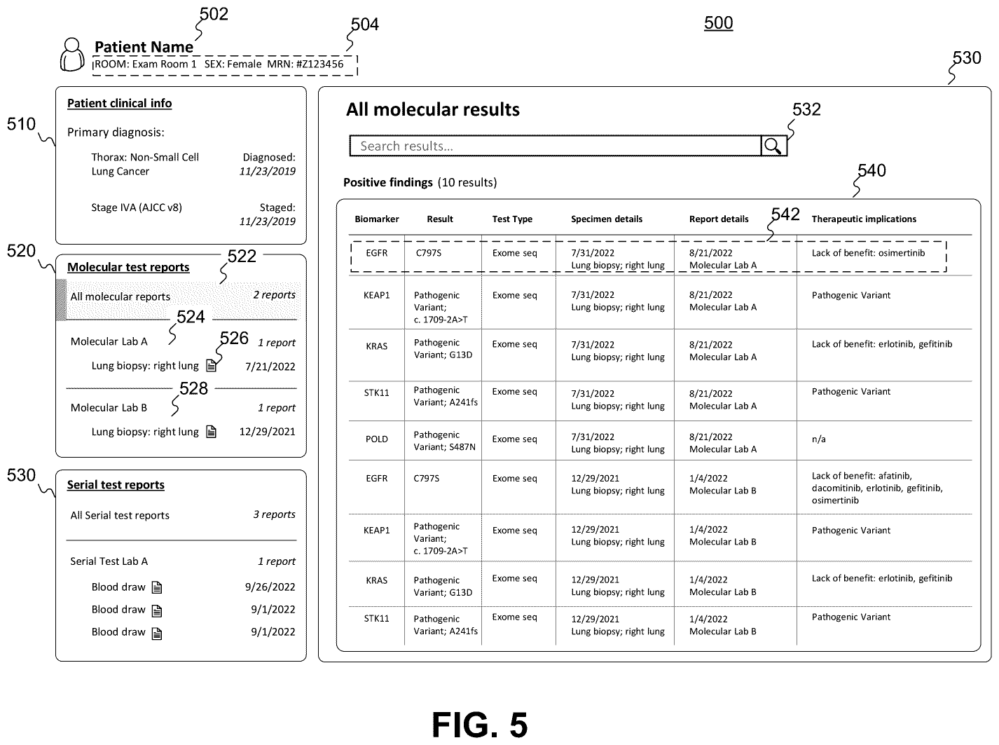

FIG. 5 illustrates an example graphical user interface 500 that may be generated, consistent with the disclosed embodiments. User interface 500 may be generated to present genomic testing data (or other test data) associated with a particular patient. For example, user interface 500 may be displayed on client device 110 to user 112. In some embodiments, user interface 500 may include information associated with a patient, such as a patient name 502 (or other identifier of the patient) and various other patient data 504. In some embodiments, user interface 500 may include a patient clinical information region 510, which may include clinical information associated with the patient. For example, clinical information region 510 may include information indicating a disease the patient has been diagnosed with, a date the diagnosis was made, a stage of the disease, a date the stage was diagnosed, or any other information associated with the treatment or condition of a patient.

In some embodiments, user interface 500 may further include one or more menu elements 520 and 530 associated with various test reports. In this example, menu element 520 may identify various molecular profiling labs from which genomic test reports associated with the patient have been received. For example, menu element 520 may include a source element 524 associated with a Molecular Lab A and a source element 528 associated with a Molecular Lab B. In this example, each of Molecular Lab A and Molecular Lab B may have provided one genomic test report, as indicated in FIG. 5. In some embodiments, menu element 520 may further include one or more document links enabling a user to access one or more genomic test reports received from a molecular profiling lab. For example, source element 524 may include a document element 526 enabling a user to view a genomic test report (or other form of test report) received from Molecular Lab A. In some embodiments, menu element 520 may further include a summary element 522, which may indicate a total number of reports associated with a patient, regardless of the source. In this example, summary element 522 may indicate the patient is associated with two genomic test reports (the sum of the number of reports from Molecular Labs A and B). As described above, the disclosed embodiments are not necessarily limited to genomic test reports and the techniques described herein may equally apply to other forms of non-standardized reports. Menu element 530 may identify various sources from which other types of test data associated with the patient have been received.

In some embodiments, summary element 522 and source elements 524 and 528 may be interactive such that selection of an element may enable a user to view additional information about the selected element. In this example, summary element 522 may be selected, which may cause graphical user interface 500 to display combined data from Molecular Lab A and Molecular Lab B. For example, graphical user interface 500 may include a region 530 for presenting information extracted from genomic test data reports received from Molecular Lab A and Molecular Lab B. For example, region 530 may include a data table 540, which may include information extracted from various genomic test reports as described above. In this example, data table 540 may include a row 542 including data associated with a particular biomarker identified in association with the patient. As shown, this may include a name of a biomarker, a biomarker result, a type of test performed, specimen details, report details, and therapeutic information. Some or all of this information may have been extracted from a genomic test report using the various mapping techniques described herein. In some embodiments, row 542 may further include an indication of the report the data was extracted from. Region 530 may further include a search element 532 enabling a user to search for particular data or types of data extracted from the genomic test reports.

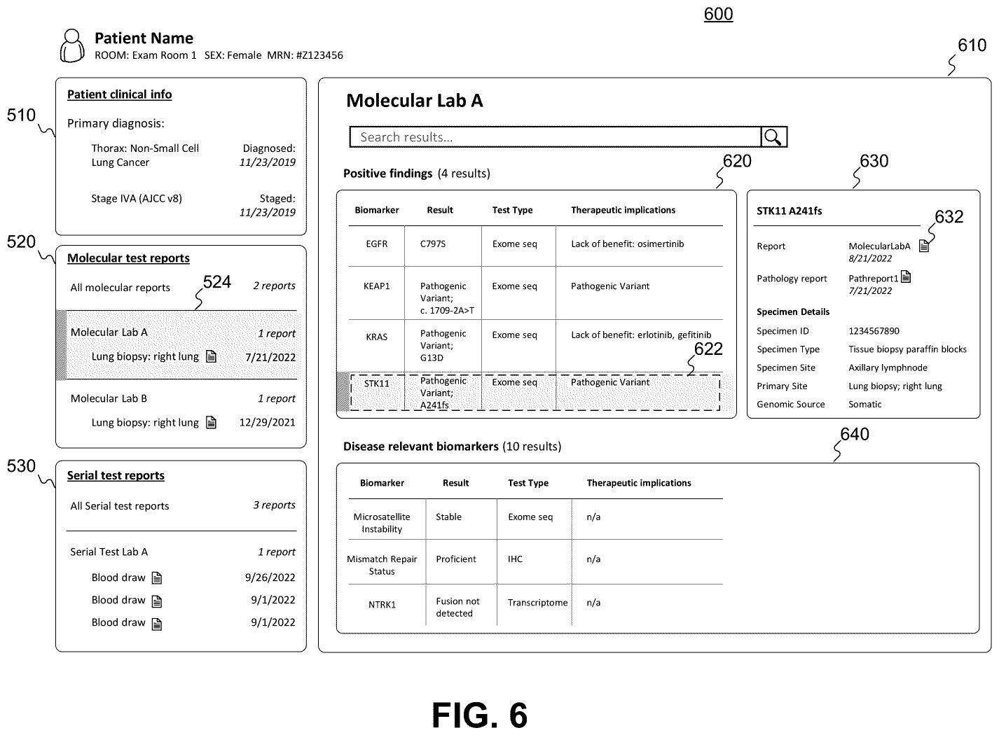

FIG. 6 illustrates an example of an additional graphical user interface 600 that may be generated, consistent with the disclosed embodiments. In this example, user interface 600 may display information associated with a particular data source. For example, user interface 600 may include a region 610 for displaying information associated with Molecular Lab A. In some embodiments, region 610 may be displayed based on a selection of a user of source element 524. For example, region 610 may be displayed in place of region 530 described above. In some embodiments, region 610 may include a data table 620, which may include information extracted from various genomic test reports received from Molecular Lab A. In this example, data table 620 may include a row 622 including data associated with a particular biomarker identified in association with the patient. As shown, this may include a name of a biomarker, a biomarker result, a type of test performed, and therapeutic information. Some or all of this information may have been extracted from a genomic test report using the various mapping techniques described herein.

In some embodiments, region 610 may further include a result element 630 for displaying information about a particular result or finding from a genomic test report. For example, result element 630 may include additional information about the result represented in row 622 (e.g., based on a selection of row 622 by a user). In some embodiments, result element 630 may include a document element 632 enabling a user to view a genomic test report that the result represented in row 622 was extracted from. For example, clicking, tapping, or otherwise selecting document element 632 may cause a report associated with row 622 to be displayed, enabling user 112 to view a report from which the result represented in row 622 was extracted. In some embodiments region 610 may further include a data table 640 summarizing results relevant to one or more diseases.

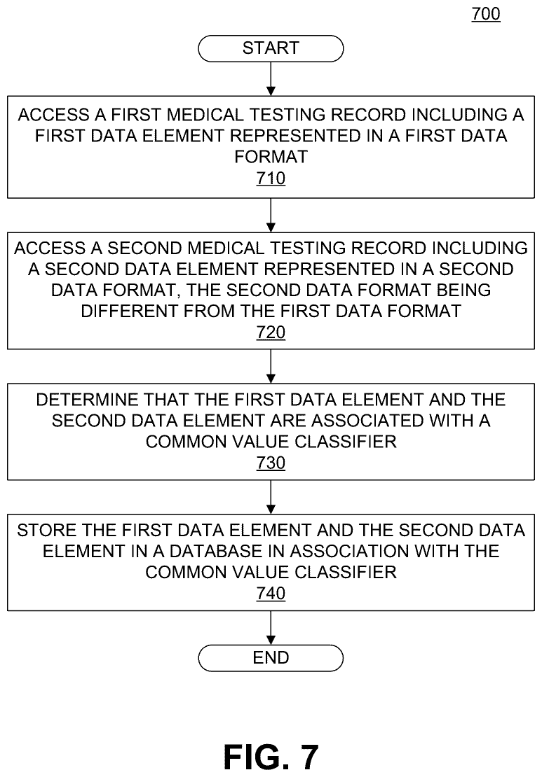

FIG. 7 is a flowchart showing an example process 700 for standardizing medical testing data, consistent with the disclosed embodiments. Process 700 may be performed by at least one processing device, such as processing engine 131, as described above. It is to be understood that throughout the present disclosure, the term “processor” is used as a shorthand for “at least one processor.” In other words, a processor may include one or more structures that perform logic operations whether such structures are collocated, connected, or dispersed. In some embodiments, a non-transitory computer readable medium may contain instructions that when executed by a processor cause the processor to perform process 700. Further, process 700 is not necessarily limited to the steps shown in FIG. 7, and any steps or processes of the various embodiments described throughout the present disclosure may also be included in process 700, including those described above with respect to FIGS. 3, 4, 5, and 6.

In step 710, process 700 may include accessing a first medical testing record including a first data element represented in a first data format. For example, step 710 may include accessing medical testing record 312, as described above. Accordingly, the first data element may correspond to data element 324, as shown in FIG. 3.

In step 720, process 700 may include accessing a second medical testing record including a second data element represented in a second data format. For example, step 720 may include accessing medical testing record 332, as described above. Accordingly, the second data element may correspond to data element 334, as shown in FIG. 3. In some embodiments, the second data format may be different from the first data format. Accordingly, steps 710 and 720 may include receiving medical testing records represented in different formats, as described herein. The first and second data formats may include a wide variety of data types and presentation formats. In some embodiments, at least one of the first data format and the second data format may include unstructured text. Alternatively or additionally, at least one of the first data format and the second data format may include structured data. For example, at least one of the first data format and the second data format may include a JSON format, as described above.

In some embodiments, the first medical testing record may be associated with a first entity and the second medical testing record may be associated with a second entity, the first entity being different from the second entity. For example medical testing record 322 may be associated with medical testing lab 320, and medical testing record 332 may be associated with medical testing lab 330, as described above.

In step 730, process 700 may include determining that the first data element and the second data element are associated with a common value classifier. For example, this may include determining data element 324 and data element 334 are associated with classifier 346, as shown in FIG. 3. As described herein, the determination of whether a data element is associated with a classifier may occur in various ways. For example, process 700 may include accessing a data structure storing a plurality of value classifiers and determining that the first data element is associated with the common value classifier may include determining, for each of the plurality of value classifiers, a degree of confidence that the first data element corresponds to the common value classifier. As another example, determining that the first data element is associated with the common value classifier may include comparing a degree of confidence associated with the common value classifier to a threshold value. For example, this may include determining a degree of confidence that data element 326 is associated with classifier 346 and comparing the degree of confidence to a threshold degree of confidence.

As another example, determining that the first data element and the second data element are associated with a common value classifier may include applying a trained machine learning model to at least one of the first medical testing record or the second medical testing record. For example, step 730 may include applying trained model 430, as described above. Accordingly the trained machine learning model may be configured to receive one or more medical testing records as an input and to generate as an output an indication of a mapping between data elements represented in the one or more medical testing records and one or more predefined value classifiers, as described further above.

In step 740, process 700 may include storing the first data element and the second data element in a database in association with the common value classifier. For example, step 740 may include storing data element 324 and data element 334 in database 132 in an associative manner with classifier 346. In some embodiments, storing the first data element and the second data element in the database may include converting the first data element and the second data element to a standardized format. For example, the standardized format may include predefined term representing a type of data associated with the first data element and the second data element. In this example, the standardized format may include the term “STK11” and may also include an indicator of whether the data element includes a positive or a negative result.

As described above, step 740 may further include storing other files, data, or information in association with the first and second data elements. For example, step 740 may further include storing the first medical testing record and the second medical testing record in the database. Alternatively or additionally, step 740 may further include storing data linking the first data element to the first medical testing record. For example, the data linking the first data element to the first medical testing record may include an indication of a location within the first medical testing record that the first data element appears.

In some embodiments, process 700 may further include presenting or making data accessible to a user. For example, the first data element and the second data element may be accessible from the database using an application programming interface (API). In some embodiments, process 700 may enable a patient summary of information extracted from medical testing reports to be displayed. For example, process 700 may further include accessing a third medical testing record including a third data element represented in a third data format. The third data format may be different from the first data format. Process 700 may further include determining that the third data element is associated with an additional value classifier different from the common value classifier; and storing the third data element in the database in association with the additional value classifier. For example, process 700 may include accessing medical testing report 312 and determining that data element 314 is associated with classifier 342, as described above. In some embodiments, process 700 may include determining that the first medical testing record and the third medical testing record are associated with a particular patient. Process 700 may further include causing display of a graphical user interface associated with the particular patient. The graphical user interface may include at least a representation of the first data element and a representation of the third data element. For example, process 700 may include causing display of graphical user interface 500 described above.

In some embodiments, the graphical user interface may be interactive. For example, the graphical user interface may include an interactive element for filtering data associated with either the first medical testing record or the third medical testing record. As another example, process 700 may include causing, based on an interaction with the representation of the first data element, display of a detail region within graphical user interface. The detail region may include information associated with the first data element extracted from the first medical testing record. For example, the detail region may correspond to result element 630 described above. The detail region may further include an element for causing display of the first medical testing record. For example, the detail region may include document element 632, as described above.

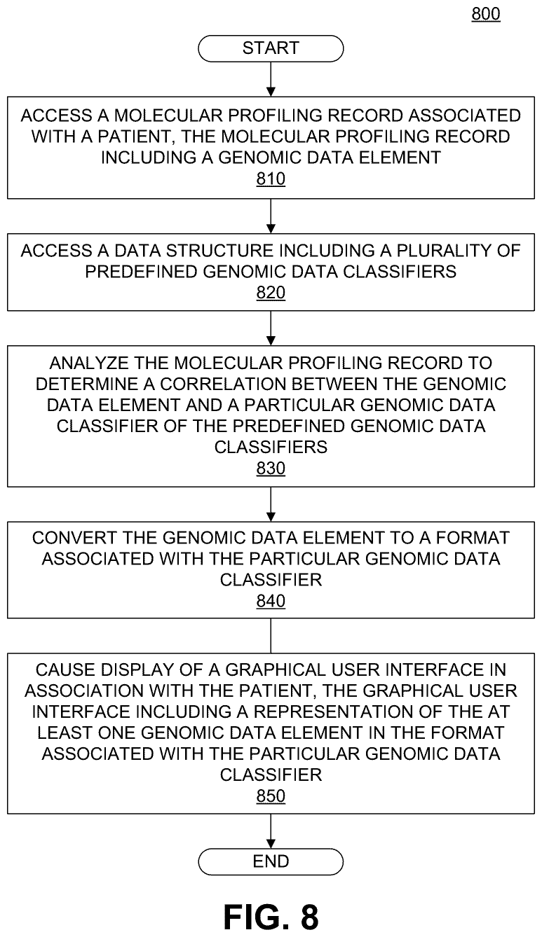

FIG. 8 is a flowchart showing an example process 800 for standardizing molecular profiling data, consistent with the disclosed embodiments. Process 800 may be performed by at least one processing device, such as processing engine 131, as described above. Process 800 is not necessarily limited to the steps shown in FIG. 8, and any steps or processes of the various embodiments described throughout the present disclosure may also be included in process 800, including those described above with respect to FIGS. 3, 4, 5, 6, and 7.

In step 810, process 800 may include accessing a molecular profiling record associated with a patient, the molecular profiling record including at least one genomic data element. For example, step 810 may include accessing medical testing record 312, as described above. Accordingly, the at least one genomic data element may correspond to data element 314, as shown in FIG. 3.

In step 820, process 800 may include accessing a data structure including a plurality of predefined genomic data classifiers. For example, step 820 may include accessing data structure 340, as described above. In this example, the plurality of predefined genomic data classifiers may include data classifiers 342, 344, and 346, and various other data classifiers, as shown in FIG. 3.

In step 830, process 800 may include analyzing the molecular profiling record to determine a correlation between the at least one genomic data element and a particular genomic data classifier of the predefined genomic data classifiers. For example, step 830 may include analyzing medical testing record 312 and determining a correlation between data element 314 and data classifier 342. As described herein, the determination of whether a genomic data element is associated with a data classifier may occur in various ways. For example, process 800 may include determining, for each of the predefined genomic data classifiers, a degree of confidence that the at least one genomic data element corresponds to the particular genomic data classifier. As another example, determining that the at least one genomic data element is associated with the particular genomic data classifier may include comparing a degree of confidence associated with the particular genomic data classifier to a threshold value. For example, this may include determining a degree of confidence that data element 314 is associated with classifier 342 and comparing the degree of confidence to a threshold degree of confidence.

As another example, determining that the at least one genomic data element is associated with the particular genomic data classifier may include applying a trained machine learning model to the molecular profiling record. For example, step 830 may include applying trained model 430, as described above. Accordingly, the trained machine learning model may be configured to receive one or more molecular profiling records as an input and to generate as an output an indication of a mapping between genomic data elements represented in the one or more molecular profiling records and one or more predefined genomic data classifiers, as described further above.

In step 840, process 800 may include converting the at least one genomic data element to a format associated with the particular genomic data classifier. For example, this may include converting data element 314 to a format associated with data classifier 342. Accordingly, various genomic data element extracted from different molecular profiling records may be stored in a standardized format, regardless of the format in which they are reported in the molecular profiling records.

In step 850, process 800 may include causing display of a graphical user interface in association with the patient. For example, step 850 may include causing graphical user interface 500 and/or graphical user interface 600 to be displayed on client device 110. The graphical user interface may include a representation of the at least one genomic data element in the format associated with the particular genomic data classifier. For example, the graphical user interface may include data table 620, which may include a row 622 including data associated with the at least one genomic data element. In some embodiments, the graphical user interface may include an interactive element displayed in association with the representation of the at least one genomic data element for causing display of the molecular profiling record. For example, step 850 may include document element 632, as described above. In some embodiments, step 850 may further include causing only a relevant portion of molecular profiling record to be displayed, causing a highlighted version of molecular profiling record to be displayed, or the like.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, 4K Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, Python, R, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A system for standardizing medical testing data, the system comprising:
at least one processor programmed to:
access a first medical testing record including a first data element represented in a first data format;
access a second medical testing record including a second data element represented in a second data format, the second data format being different from the first data format;
determine that the first data element and the second data element are associated with a common value classifier; and
store the first data element and the second data element in a database in association with the common value classifier.

2. The system of claim 1, wherein the first medical testing record is associated with a first entity and the second medical testing record is associated with a second entity, the first entity being different from the second entity.

3. The system of claim 1, wherein determining that the first data element and the second data element are associated with a common value classifier includes applying a trained machine learning model to at least one of the first medical testing record or the second medical testing record, the trained machine learning model being configured to receive one or more medical testing records as an input and to generate as an output an indication of a mapping between data elements represented in the one or more medical testing records and one or more predefined value classifiers.

4. The system of claim 1, wherein at least one of the first data format and the second data format include unstructured text.

5. The system of claim 1, wherein at least one of the first data format and the second data format include structured data.

6. The system of claim 1, wherein the first data element and the second data element are accessible from the database using an application programming interface (API).

7. The system of claim 1, wherein storing the first data element and the second data element in the database includes converting the first data element and the second data element to a standardized format.

8. The system of claim 7, wherein the standardized format includes predefined term representing a type of data associated with the first data element and the second data element.

9. The system of claim 1, wherein the at least one processor is further programmed to store the first medical testing record and the second medical testing record in the database.

10. The system of claim 1, wherein the at least one processor is further programmed to store data linking the first data element to the first medical testing record.

11. The system of claim 10, wherein the data linking the first data element to the first medical testing record includes an indication of a location within the first medical testing record that the first data element appears.

12. The system of claim 1, wherein the at least one processor is further programmed to access a data structure storing a plurality of value classifiers and wherein determining that the first data element is associated with the common value classifier includes determining, for each of the plurality of value classifiers, a degree of confidence that the first data element corresponds to the common value classifier.

13. The system of claim 12, wherein determining that the first data element is associated with the common value classifier includes comparing a degree of confidence associated with the common value classifier to a threshold value.

14. The system of claim 1, wherein the at least one processor is further programmed to:
access a third medical testing record including a third data element represented in a third data format, the third data format being different from the first data format;
determine that the third data element is associated with an additional value classifier, the additional value classifier being different from the common value classifier; and
store the third data element in the database in association with the additional value classifier.

15. The system of claim 14, wherein the at least one processor is further programmed to determine that the first medical testing record and the third medical testing record are associated with a particular patient.

16. The system of claim 15, wherein the at least one processor is further programmed to cause display of a graphical user interface associated with the particular patient, the graphical user interface including at least a representation of the first data element and a representation of the third data element.

17. The system of claim 16, wherein the at least one processor is further programmed to cause, based on an interaction with the representation of the first data element, display of a detail region within graphical user interface, the detail region including information associated with the first data element extracted from the first medical testing record.

18. The system of claim 17, wherein the detail region further includes an element for causing display of the first medical testing record.

19. The system of claim 16, wherein the graphical user interface includes an interactive element for filtering data associated with either the first medical testing record or the third medical testing record.

20. A computer-implemented method for standardizing medical testing data, the method comprising:
accessing a first medical testing record including a first data element represented in a first data format;
accessing a second medical testing record including a second data element represented in a second data format, the second data format being different from the first data format;
determining that the first data element and the second data element are associated with a common value classifier; and
storing the first data element and the second data element in a database in association with the common value classifier.

21. A non-transitory computer readable medium containing instructions that when executed by at least one processor, cause the at least one processor to perform a method for standardizing medical testing data, the method comprising:
accessing a first medical testing record including a first data element represented in a first data format;
accessing a second medical testing record including a second data element represented in a second data format, the second data format being different from the first data format;
determining that the first data element and the second data element are associated with a common value classifier; and
storing the first data element and the second data element in a database in association with the common value classifier.

22. A system for standardizing molecular profiling data, the system comprising:
at least one processor programmed to:
access a molecular profiling record associated with a patient, the molecular profiling record including at least one genomic data element;
access a data structure including a plurality of predefined genomic data classifiers;
analyze the molecular profiling record to determine a correlation between the at least one genomic data element and a particular genomic data classifier of the predefined genomic data classifiers;
convert the at least one genomic data element to a format associated with the particular genomic data classifier; and
cause display of a graphical user interface in association with the patient, the graphical user interface including a representation of the at least one genomic data element in the format associated with the particular genomic data classifier, wherein the graphical user interface includes an interactive element displayed in association with the representation of the at least one genomic data element for causing display of the molecular profiling record.
